(19) United States
(12) Patent Application Publication
(10) Pub. No.: US 2007/0265649 A1
(43) Pub. Date: Nov. 15, 2007

(54) Epithelial delaminating device

(76) Inventor: Edward Perez, Palo Alto, CA (US)

Correspondence Address: E. THOMAS WHEELOCK, P.O. BOX 61168, PALO ALTO, CA, 94306, US

(21) Appl. No.: 11/640,630

(22) Filed: Dec. 18, 2006

Related U.S. Application Data
(63) Continuation of application No. PCT/US05/21347, filed on Jun. 16, 2005.
(60) Provisional application No. 60/580,430, filed on Jun. 16, 2004.

(51) Int. Cl. A61F 9/00

(52) U.S. Cl. 606/166; 606/161

(57) ABSTRACT

The described device is useful in the field of opthalmology. The devices and methods for using it involve separating or lifting corneal epithelium from the eye in a substantially continuous layer to form a flap or pocket. In particular, the devices generally utilize a non-cutting, oscillating separator or dissector that is configured to separate the epithelium at naturally occurring cleavage surfaces in the eye, particularly between the epithelium and the corneal stroma (Bowman's membrane), specifically separating in the region of the lamina lucida, the separator or dissector having a structure that oscillates at that cleavage surface interface during the dissection step. The separated epithelium may be lifted or peeled from the surface of the eye to form an epithelial flap or pocket. The epithelium may then be replaced on the cornea after a refractive procedure or placement of an ocular lens on the eye.

### RELATED APPLICATIONS

This is a continuation of International Application PCT/US2005/021347, filed Jun. 16, 2005, which in turn derives benefit from U.S. Provisional Application 60/580,430, filed Jun. 16, 2004.

### FIELD

The described device is useful in the field of opthalmology. The devices and methods for using it involve separating or lifting corneal epithelium from the eye in a substantially continuous layer to form a flap or pocket. In particular, the devices generally utilize a non-cutting, oscillating separator or dissector that is configured to separate the epithelium at naturally occurring cleavage surfaces in the eye, particularly between the epithelium and the corneal stroma (Bowman's membrane), specifically separating in the region of the lamina lucida, the separator or dissector having a structure that oscillates at that cleavage surface interface during the dissection step. The separated epithelium may be lifted or peeled from the surface of the eye to form an epithelial flap or pocket. The epithelium may then be replaced on the cornea after a refractive procedure or onto an ocular lens after placement of that ocular lens on the eye.

### BACKGROUND

Refractive surgery refers to a set of surgical procedures that change the native optical or focusing power of the eye. These changes alleviate the need for glasses or contact lenses that an individual might otherwise be dependent on for clear sight. The majority of the focusing power in the human eye is dictated by the curvature of the air-liquid interface, where there is the greatest change in the index of refraction. This curved interface is the outer surface of the cornea. The refractive power of this interface accounts for approximately 70% of the total magnification of the eye. Light rays that make up the images we see pass through the cornea, the anterior chamber, the crystalline lens, and the vitreous humor before they are focused on the retina to form an image. It is the magnifying power of this curved, air-corneal interface that provided the field of refractive surgery with the opportunity to surgically correct visual deficiencies.

Initial refractive surgical procedures corrected nearsightedness by flattening of the curvature of the cornea. The first largely successful procedure was called radial keratotomy (RK). RK was widely used during the 1970's and early 1980's where radially oriented incisions were made in the periphery of the cornea. These incisions allowed the peripheral cornea to bow outwards, consequently flattening the central optical zone of the cornea. This was fairly easy and thus, popular, but it rarely did more than lessen one's dependency on glasses or contract lenses.

A largely flawed and failed procedure called epikeratophakia was developed in the era of RK. It is now essentially an academic anomaly. Epikeratophakia provided a new curvature to the outer curvature of the cornea by grafting onto the cornea a thin layer of preserved corneal tissue. Lyophilization is the preservation method used in epikeratophakia where the cornea is freeze-dried. The tissue is not acellularized but is rendered non-living. During the process of freeze drying, the cornea is also ground to a specific curvature.

The epikeratophakia lens was placed into the eye surgically. An annular 360° incision was placed into the cornea after completely removing the epithelium from where the epikeratophakic lens would sit. The perimeter of this lens would be inserted into the annular incision and held in place by a running suture. There were several problems with epikeratophakia: 1) the lenses remained cloudy until host stromal fibroblasts colonized the lens, which colonization possibly could take several months; 2) until migrating epithelium could grow over the incision site onto the surface of the lens, the interrupted epithelium was a nidus for infection; and 3) epithelium healing onto the surgical site sometimes moved into the space between the lens and the host cornea. Currently, epikeratophakia is limited in its use. It is now used in pediatric aphakic patients who are unable to tolerate very steep contact lenses.

Major industrial research efforts tried to produce a synthetic version of the epikeratophakic graft called the synthetic onlay in a synthetic epilens. Different synthetic polymers were used (hydroxyethylmethacrylate, polyethylene oxide, lidofilcon, polyvinyl alcohol). Hydrogels of these materials normally did not have a surface that was readily conducive to epithelial cells growing and adhering onto these synthetic surfaces. This was one of the major setbacks of synthetic onlays. Epithelial cells could not adequately heal onto these lenses.

Another problem with these synthetic lenses is that they did not adhere well to the surface of the eye. Conventional suturing was difficult and the use of biological glues was also flawed. Glues were not ideally biocompatible in the cornea.

Lastly, the permeability of these hydrogels was significantly limiting. Living epithelial cells on the surface had difficulty achieving adequate nutrition. Corneal epithelial nutritional flow is from the aqueous humor through the cornea out to the epithelial cells. In the end, industrial efforts failed to develop an adequate synthetic epikeratophakic lens.

Around the mid 1990's procedures that sculpt the cornea with lasers were sufficiently successful that they began to replace radial keratotomy. The first generation of laser ablation of the cornea was called photorefractive keratectomy (PRK). In PRK, an ablative laser (e.g., an excimer laser) is focused on the cornea to sculpt a new curvature into the surface. In PRK, the epithelium is destroyed when achieving a new outer surface curve. Over the ensuing post-operative days, the epithelium has to grow or heal back into place. This epithelial healing phase was problematic for most patients since the epithelially denuded and ablated cornea was painful. It is also initially difficult to see, and this “recuperative time” can last from days to a week or more.

A subsequent variation of PRK corneal laser ablation, LASIK, has become very popular. The LASIK procedure, also known as laser in situ keratomileusis, is synonymous in the public mind with laser vision correction. In LASIK, an outer portion (or chord-like lens-shaped portion) of the cornea (80 to 150 microns thick) is surgically cut from the corneal surface. This is performed by a device called a microkeratome. The microkeratome is a device which cuts a circular flap from the surface of the cornea which remains hinged at one edge. This flap is reflected back and an ablative (excimer) laser is used to remove or to reform a portion of the exposed surgical bed. The flap is laid back into place. When this flap is laid back into place, the cornea achieves a new curvature because the flap conforms to the laser-modified surface. In this procedure, epithelial cells are not removed or harmed. The epithelial cells have simply been incised at the edge of this flap. When the flap is placed back onto the corneal bed, the epithelium heals back at the incision site. There is essentially no recuperative time and the results are almost immediate. Because there is very little surgical time (15 minutes for each eye) and because there are lasting and very accurate results, LASIK is currently considered the premier manner of performing refractive surgery.

The newest technique being evaluated in high volume refractive surgical practices and in some academic centers is a procedure called Laser Assisted Subepithelial Keratomileusis (LASEK). In LASEK, a “flap” is made of only epithelium. This layer of epithelium is lifted off the cornea in a manner similar to LASIK. The ablative laser is focused just on the surface of the denuded cornea (in the same manner as was done with PRK). However, this epithelial flap is left intact, i.e., epithelium is not destroyed. It is simply rolled back into place after formation of the re-curved anterior portion of the cornea, resulting in much less recuperative time than with PRK. Current methods of LASEK are not as good as LASIK but the results are better than with PRK.

The corneal epithelium is a multilayered epithelial structure typically about 50 μm in thickness. It is non-cornified. The outer cells are living, although they are squamous in nature. The basal epithelial cells are cuboidal and sit on the stromal surface on a structure known as Bowman's membrane. The basal cell layers is typically about 1 mil thick (0.001″). The basal cells produce the same keratins that are produced in the integument, i.e., skin. The basal epithelial cells express keratins 5 and 14 and have the potential to differentiate into the squamous epithelial cells of the corneal epithelium that produce keratins 6 and 9. The corneal epithelium has a number of important properties: 1) it is clear; 2) it is impermeable; 3) it is a barrier to external agents; and 4) it is a highly innervated organ. Nerves from the cornea directly feed into the epithelium, and thus, defects of this organ produce pain.

Epithelial cells are attached side-to-side by transmembrane molecules called desmosomes. Another transmembrane protein, the hemidesmosome, connects to collagen type 7 and is present on the basolateral surface of basal epithelial cells. Hemidesmosomes anchor epithelium to the underlying collagenous portion of the stroma. The junction between the epithelium and corneal stroma is referred to as basement membrane zone (BMZ).

When LASEK is performed, a physical well is placed or formed on the epithelium and filled with a selection of 20 percent ethanol and balanced salt solution. Contact with the solution causes the epithelial cells to lose their adherence at the BMZ, most likely by destroying a portion of that cell population. The epithelium is then raised by pushing the epithelium, e.g., with a Week sponge, in a manner similar to striping a wall of paint. The exposed collagenous portion of the corneal stroma is then ablated to reshape its surface. A weakened epithelium is then rolled back into place to serve as a bandage. However, this “bandage” fails to restore the epithelium to its original state, i.e., it does not preserve the integrity of the epithelium, thereby reducing its clarity, impermeability to water, and barrier function. Furthermore, the ability of the epithelium to adhere to the corneal stromal surface is impaired.

U.S. Pat. Nos. 6,099,541 and 6,030,398 to Klopotek describe an microkeratome apparatus and method for cutting a layer of corneal epithelium to prepare the eye for LASIK or other reshaping procedures. The epithelium, if replaced, is attached using surgical techniques.

None of the cited references shows or suggests my invention as described herein.

### REFERENCES

Kiistala, U. (1972). “Dermal-Epidermal Separation. II. External Factors in Suction Blister Formation with Special Reference to the Effect of Temperature,” Ann Clin Res 4(4):236-246.
Azar et al. (2001). “Laser Subepithelial Keratomileusis: Electron Microscopy and Visual Outcomes of Flap Photorefractive Keratectomy,” Citrr Opin Opthalmol 12(4):323-328.
Beerens et al. (1975). “Rapid Regeneration of the Dermal-Epidermal Junction After Partial Separation by Vacuum: An Electron Microscopic Study,” J Invest Dermatol 65(6):513-521.
Willsteed et al. (1991). “An Ultrastructural Comparison of Dermo-Epidermal Separation Techniques,” J Cutan Pathol 18(1):8-12.
van der Leun et al. (1974). “Repair of Dermal-Epidermal Adherence: A Rapid Process Observed in Experiments on Blistering with Interrupted Suction,” J Invest Dermatol 63 (5): 397401.
Katz S I. (1984). “The Epidermal Basement Membrane: Structure, Ontogeny and Role in Disease,” Ciba Found Symp 108:243-259.
Green et al. (1996). “Desmosomes and Hemidesmosomes: Structure and Function of Molecular Components,” FASEB J 10(8):871-881.

### SUMMARY

The description includes mechanical non-cutting devices and methods to form a separation of the epithelium from the eye or to lift a generally continuous layer of epithelium from its supporting underlying structure. The epithelial delaminator is used to create an epithelial flap or a pocket. The flap or pocket may be used in conjunction with a refractive surgical procedure or with placement of refractive lens.

The epithelial delaminator may be mechanical in nature. Such mechanical delaminators lift epithelium in a generally continuous layer from the anterior surface of the eye by application of a dissecting, non-cutting, mechanical force. Mechanical delaminators specifically include blunt dissectors and wire-based dissectors having wires that are passive or active as applied to the eye. Of particular interest here are mechanical delaminators that are in the nature of vibrating or oscillating spatulas and are able to form epithelial pockets and flaps with reasonable ease.

Furthermore, the method of this invention may be used variously to de-epithelialize the cornea in preparation for a reshaping procedure such as LASEK or to form a pocket for inclusion of a contact lens.

### BRIEF DESCRIPTION OF THE DRAWINGS

FIG. IA is a partial top view of an oscillating tip useful in separating the corneal epithelium.

FIG. IB is a partial side view of the FIG. IA device.

FIG. 1C is an axial, cross-sectional view of the FIG. IA device.

FIGS. 2A, 2B, and 2C are partial top views of various oscillating tips.

FIGS. 3A, 3B, and 3C are partial side views of various oscillating tips.

FIGS. 4A and 4B show before and after top views of one way of forming a delaminator tip.

FIG. 5A is a partial, cutaway, perspective view of a hand-held version of the mechanical epithelial delaminator separator system showing the overall placement of its components and its operation.

FIG. 5B shows a partial side-view of one way of connecting the blade to the motor.

FIGS. 6A, 6B, and 6C show perspective views of orientation plates as used in this system.

FIG. 7A shows a partial top-view of a dissector delaminator having an oscillating, rotating motion at the dissector tip.

FIG. 7B shows a partial side-view of the delaminator shown in FIG. 7A.

FIGS. 8, 9, and 10 show top views of dissectors having various tip motions.

### DETAILED DESCRIPTION

For any integument surface such as the skin, respiratory epithelium, gut epithelium, and cornea, there is an epithelial cell layer that is adherent to an underlying basement membrane. When epithelium is separated from its basement membrane and underlying collagenous tissue, a subepithelial blister is formed. In general, gross separation less than 1 mm in diameter is known as vesiculation and separation greater than 1 millimeter in diameter, a true blister.

A continuous layer of corneal epithelium may be separated from or lifted from the anterior surface of the eye by applying various mechanical forces to this anterior surface, or to the basal cell layer, or to the junction between the basal cell layer and the Bowman membrane (the “lamina lucida”). The term “continuous” as used herein means “uninterrupted”. The term “mechanical force” as used herein refers to any physical force produced by a person, instrument, or device. Examples of mechanical forces include suction, shearing, and blunt forces.

Mechanical forces are applied to epithelium such as corneal epithelium by epithelial delaminators. As used herein, the term “epithelial delaminator” refers to any instrument or device that separates epithelium from the basement membrane by application of a mechanical force. Epithelium may also be separated from or lifted from the anterior surface of the eye by contacting the surface with a chemical composition that induces separation of the epithelium from the underlying stroma.

### Oscillating or Vibrating Mechanical Epithelial Delaminators

In a first variation of this mechanical epithelial delaminator, the delaminator comprises a blunt, spatula-like delaminator tool (100) as is seen in FIG. IA. Typically, this tool (100) will be attached to a driver motor in such a way that the blunt tip (102) moves it a repetitive, oscillatory motion (104) that easily separates corneal epithelium from its underlying tissue without cutting that stromal tissue. In at least one variation of the device, the tip (102) moves in at least one of a side-to-side motion and an up-and-down motion. The delaminator tool (100) may be modestly cupped in the vicinity of the end (102) as may be better seen in FIGS. IB and 1C. One method for forming such a cupped end will be discussed below.

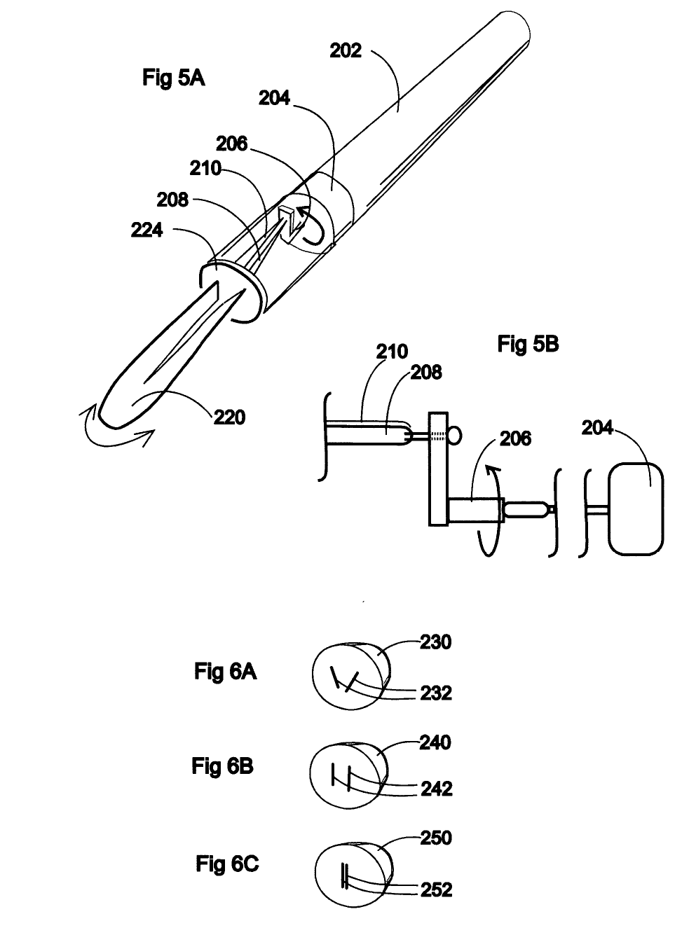

The oscillatory motion (104) of the tip (102) may be produced by moving the two arms (106, 108) of the tool (100) back-and-forth as shown by arrows (110, 112). The movement of the two arms (106, 108) should be “out of phase” to cause the oscillatory motion (104). That is to say: arm (106) should be pushed while arm (108) is pulled or is stationary and arm (108) should be pushed while arm (106) is pulled or is stationary. Further, the motions imparted to the two arms from the distal ends of the arms (106, 108) by the rotational member discussed below with respect to FIGS. 5A and 5B is much more complex than is simply stated here and causes simultaneous multi-axis motions at the tip, but is included in the motion description provided just above.

The end or blunt tip (102) may be of the specific shape and bluntness shown in FIGS. IA, IB, and 1C with good results, but the tip (102) may be of other shapes, e.g., with a point or with a straight end or circular form, and other levels of bluntness, e.g., with additional sharpness, e.g., approaching a knife edge. Such choices are left to the designer at the time this teaching is taken and applied to the design of a tool for accomplishment of a specific task or procedure. For instance, the choice of a wide tool (100) with a blunt tip might be the best choice for the creation of a large epithelial pocket and installation of a large contact lens in that pocket.

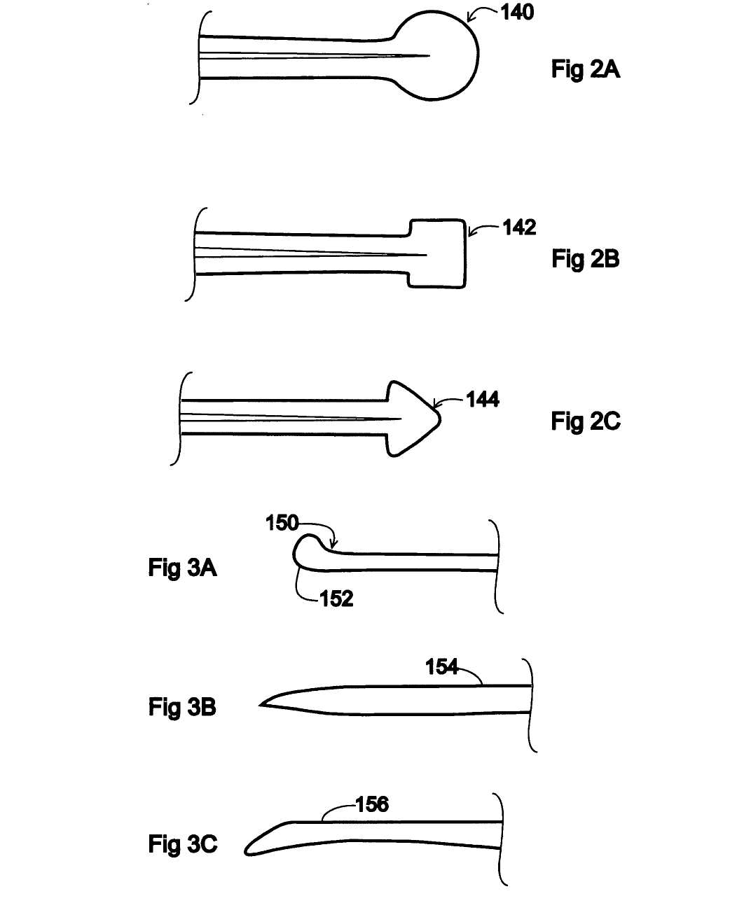

FIGS. 2A, 2B, and 2C show examples of tip shape variations and FIGS. 3A, 3B, and 3C show tip sharpness variations.

FIG. 2 A shows a top view of a round tip (140) that may be used, for instance, when separating large areas of epithelium or scarred or previously diseased epithelium. The larger area may be considered as more gentle in many circumstances.

FIG. 2B shows a top view of a straight ended tip (142) that may be used, for instance, in the instance discussed just above.

FIG. 2C shows a top view of an arrow-shaped tip (144). Such a tip may be useful in initially traversing a tougher epithelium or in instances where a tip with greater control is needed.

FIG. 3A shows a side view of a tip (150) having a distal bulb (152). In addition to initial separation of the epithelium from the corneal stroma, the tip may be used in expanding an epithelial pocket previously or contemporaneously formed.

FIG. 3B shows a side view of a tip (154) having a comparatively sharp tip.

FIG. 3C shows a side view of a tip (156) having a blunt but asymmetrical tip.

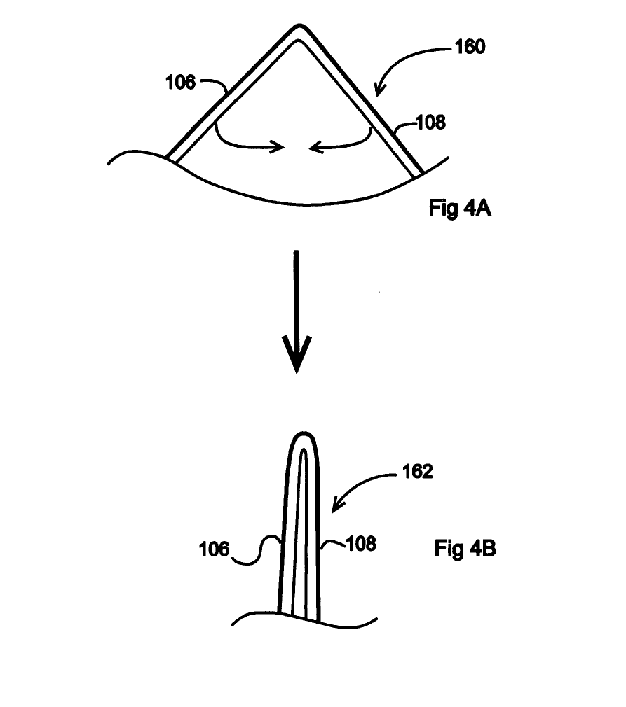

The delaminating dissector tips discussed above may be formed in a variety of ways, but a desirable way is by simply forming a “pre-form” or “pre-tip” and then bending the tip into the final desired shape. For instance, the tip shown in FIG. IA may be formed from a “pre-tip” (160) as found in FIG. 4A by moving the arms (106, 108) toward each other, e,g,. by bending into the form (162) shown in FIG. 4B. Since the tip is made from a springy material such as a stainless steel or a super-elastic alloy such as “nitinol,” the cupping mentioned above is inherently formed.

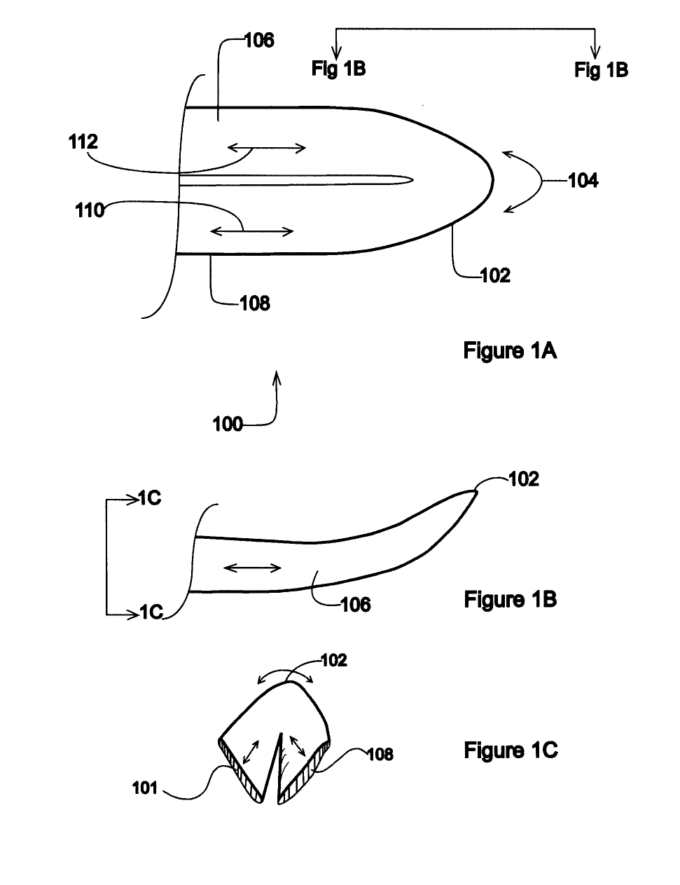

The oscillatory motion mentioned above with respect to FIGS. 1A-IC may be provided a driver such as shown (in a summary or schematic fashion) in FIGS. 5A and 5B. These devices likely will be used in manual surgery and consequently will often be formed with a handle. The variation of the driver assembly (200) shown in FIG. 5 may be handled in the fashion of a scalpel.

Driver assembly (200) comprises a battery pack (202) driving a rotary electric motor (204). The rotary motor turns a rotating member, such as a arm or disk, (206) attached to the arm segments (208, 210) of the tip (212). As the motor (204) and rotating member (e.g., arm or disk) (206) rotates, the attached arm segments (208, 210) follow it but are allowed to rotate freely with respect to the rotating arm (206). In this way, the arm segments (208, 210) maintain a specific orientation to the driver assembly as a whole. The arm segments (208, 210) pass through an orientation plate (214) and terminate at the tip (220). The rotation of the motor (204) through the rotating arm (206) moves the two arm segments (208, 210) in a coordinated fashion and causes the “out of phase” motion or “non-simultaneous” motion for the arm segments mentioned above. That is to say: the movable arm segments (208, 210) have distal ends remote from the movable tip (220) that, when attached to the rotating member (206) cause those distal ends to have a rotational motion such that the movable arm segments (208, 210) are moved, but are not simultaneously moved in the same relative direction with respect to each other, at the same time, the movable arm segments (208, 210) cooperate and cause at least one of a side-to-side motion and an up-and-down motion at the movable tip (220).

The orientation plate (214) provides a relatively constant form and physical location to the tip (220).

As shown in FIGS. 6A, 6B, and 6C, the slots in the orientation plates may be of a number of configurations. FIG. 6A shows a configuration plate (230) having canted slots (232). FIG. 6B shows a configuration plate (240) having parallel, spaced-apart slots (234). FIG. 6C shows a configuration plate (250) having parallel, close slots (252).

The described mechanical epithelial delaminators may also be considered to be blunt dissectors. Blunt dissectors have non-cutting surfaces that are appropriate for placement between the epithelium and the collagenous stromal tissue. As used herein, the term “non-cutting” means that the blunt dissector does not have the ability to incise into the stroma of the cornea when used with normal force. I believe that my blunt dissectors separate the epithelium from the stromal layers of the cornea in the basal membrane zone at the natural point of weakest attachment, i.e., the lamina lucida. The so-separated epithelium does not contain substantial amounts of corneal stromal tissue, or for purposes of this invention, does not contain any more than an insubstantial amount of the stromal tissue when the procedure is practiced on “normal” eyes (those having no artifacts due to injury or to disease). The so-separated epithelium does not contain Collagen Type I or Type III as may be found in the stromal tissues.

I have found that delaminator tips made according to this description may be made of springy materials, as discussed above, having a thickness similar to the thickness of the basal cell layer, e.g., about ½ mil to 3.5 mils. (0.0005 to 0.0035″), but often about 1.0 mil to 3.0 mils (0.001 to 0.003″). A thickness near 2.0 mils is excellent.

Although the procedure here is normally used to dissect a substantially intact sheet of the epithelium, i.e., the portion of the epithelium that passes to the anterior side of the dissector is continuous, the device may be used in less elegant ways. For instance, the dissector may be used to remove selected portions of that membrane. Indeed, when this device is used in conjunction with a LASEK procedure, the epithelium may be removed in the form of a soft flap allowing for ease of replacement or re-positioning once any corneal laser remodeling is completed. This dissector may be used to form an epithelial pocket.

In some instances it may be desirable to also apply heat to the anterior surface of the eye to enhance the mechanical epithelial delamination.

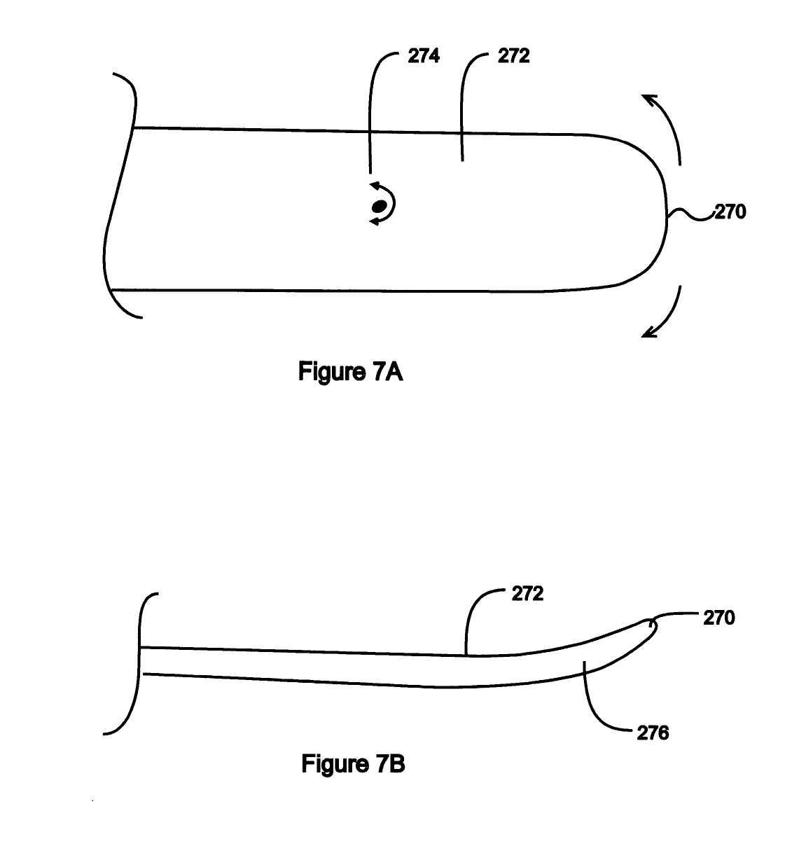

Additional variations of the dissector device and of the motions at their distal tip are shown in FIGS. 7A, 8, 9, and 10.

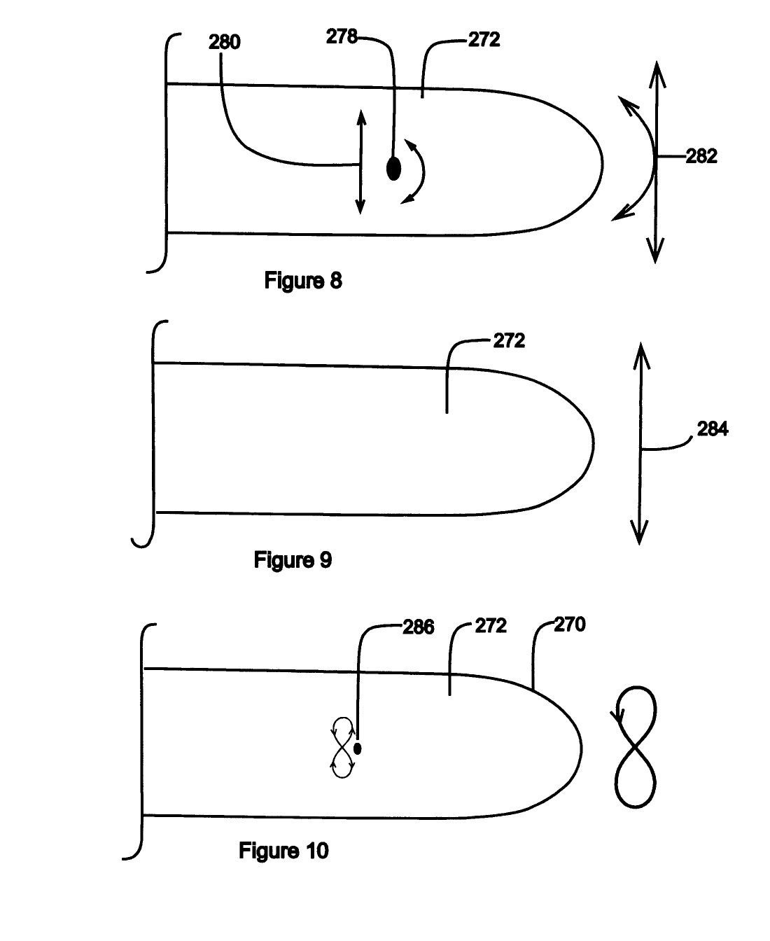

FIG. 7A shows a simple blunt tip (270) on a dissector (272). Again, the tip (270) is not sufficiently sharp to cut into the cornea. This particular variation includes a center of rotation (274) that may itself be moved longitudinally (as may be seen in FIG. 10) or side-to-side (as shown in FIG. 8). This variety of motions allows the dissector described here to be used for a variety of variously difficult and simple epithelial delamination procedures.

FIG. 7B shows a side view of the delaminating dissector (272) with its suitably blunt tip (270). It may be observed that the distal portion of dissector (272) includes a fairly gentle curve (276) to allow its easy use upon the corneal epithelium.

FIG. 8 shows the dissector blade (272) having both a center of rotation (278) about which the blade oscillates and rotates. The center of rotation (278) also translates from side-to-side (280) to provide a complex, rotating, translating movement (282) at the distal tip.

FIG. 9 depicts a dissector blade (272) that simply oscillates in a linear fashion (284) from side-to-side without including any longitudinal motion.

Finally, FIG. 10 shows a dissector blade (272) having an axis of oscillatory rotation (286) that is moved in a figure-eight movement. This allows the tip of the blade (270) to move both side-to-side and (slightly) along the longitudinal axis of the blade (272).

The epithelial delaminating methods herein described may also be used in conjunction with corneal reshaping procedures or procedures that involve placement of ocular lens devices on the surface of the eye. Specifically, the disclosed procedure may be used to prepare an epithelial pocket or a flap, often with an attached hinge. A suitable ocular lens may then be placed on the stromal surface and the epithelial flap replaced over the lens. One such suitable ocular lens device to be used with the present invention is described in Application No. PCT/US01/22633 which is herein incorporated by reference in its entirety.

Similarly, a corneal reshaping procedure may be performed and the corneal flap replaced. The structure and physiologic properties for my invention, as well as certain of the benefits particular to the specific variations of this epithelial delaminating device, have been described. This manner of describing the invention should not, however, be taken as limiting the scope of the invention in any way.

I claim as my invention:

1. A device for separating epithelium from an eye having a cornea with epithelium and a stroma, the device comprising an oscillating epithelial delaminator member configured to apply a mechanical force beneath that epithelium to separate the epithelium from the stroma without cutting that stroma, said separated epithelium being substantially free of Collagen Type I and Collagen Type III.

2. The device of claim 1 wherein the oscillating epithelial delaminator member comprises a spatula-like or substantially flat member formed into a form having a small hollow.

3. The device of claim 1 wherein the oscillating epithelial delaminator member comprises a movable tip having a side-to-side axis and an up-and-down axis and movable arms that, configured so that when the arms are moved, but not simultaneously moved in the same relative direction at the same time, the arms cooperate to cause at least a side-to-side motion in the movable tip.

4. The device of claim 3 wherein the movable arms are configured so that when the arms are moved, but not simultaneously moved in the same relative direction at the same time, the arms cooperate further to cause at least an up-and-down motion in the movable tip.

5. The device of claim 3 wherein the movable arms are distally moved in a rotational motion such that when the arms are moved, but not simultaneously moved in the same relative direction at the same time, the arms cooperate and cause at least one of a side-to-side motion and an up-and-down motion at the movable tip.

6. The device of claim 3 wherein the movable arms have distal ends remote from the movable tip, the device further comprising a rotating member causing the distal ends of the movable arms to have a rotational motion such that when the are moved, but not simultaneously moved in the same relative direction at the same time, the arms cooperate and cause at least one of a side-to-side motion and an up-and-down motion at the movable tip.

7. The device of claim 3 further comprising an orientation plate having openings through which the movable arms pass.

8. The device of claim 1 wherein the oscillating epithelial delaminator member comprises a movable tip having a side-to-side axis and an up-and down axis and is configured to move in at least one of a side-to-side motion and an up-and-down motion.

9. The device of claim 1 wherein the oscillating epithelial delaminator comprises a movable tip having a side-to-side axis and a back-and-forth longitudinal axis and is configured to move in at least one of a side-to-side motion and a back-and-forth motion

10. The device of claim 1 wherein the oscillating epithelial delaminator member is configured to separate the epithelium in at least one continuous portion.

11. The device of claim 1 wherein the oscillating epithelial delaminator member is configured to separate the epithelium and form an epithelial pocket.

12. The device of claim 1 wherein the oscillating epithelial delaminator member is configured to separate the epithelium and form an epithelial flap.

13. A method for lifting epithelium from an eye having a cornea with an epithelium and stroma, comprising the steps of:
placing an epithelial delaminator member of any of claims 1-12 beneath the epithelium, and
moving the epithelial delaminator member to apply a mechanical force beneath the epithelium with a force sufficient to separate the epithelium in a continuous layer from the stroma, but not to cut the stroma.

14. The method of claim 13 where the step of applying a mechanical force comprises a step of forming an epithelial pocket.

15. The method of claim 13 where the step of applying a mechanical force comprises a step of forming an epithelial flap.

16. The method of claim 13 where the step of applying a mechanical force comprises a step of peeling the epithelial flap to expose the stroma.

17. The method of claim 13 further comprising the step of performing a surgical step on the stroma.

18. The method of claim 17 where the surgical step comprises reshaping the stroma.

19. The method of claim 18 further comprising the step of replacing the flap on the stroma.

20. The method of claim 17 further comprising the step of placing an ocular lens on the stroma.

21. The method of claim 20 further comprising the step of replacing the flap on the stroma.

22. The method of claim 13 where the step of applying a mechanical force comprises a step of forming an epithelial pocket or flap.

23. The method of claim 22 further comprising the step of placing an ocular lens on the stroma beneath the epithelium.

24. A method for forming an attached epithelium flap or pocket on an eye having a cornea with an epithelium and stroma, comprising the steps of:
placing an epithelial delaminator member beneath the epithelium and
moving the epithelial delaminator member to apply a mechanical force beneath the epithelium with a force sufficient to form a separated epithelial tissue, an epithelial flap, or epithelial pocket attached with epithelial tissue to the stroma, but not to cut the stroma.

25. The method of claim 24 where the step of applying a mechanical force comprises a step of forming a separated epithelial tissue.

26. The method of claim 24 where the step of applying a mechanical force comprises a step of forming an epithelial flap.

27. The method of claim 24 where the step of applying a mechanical force comprises a step of forming an epithelial pocket.

28. The method of claim 24 where the step of applying a mechanical force comprises a step of peeling the epithelial flap to expose the stroma.

29. The method of claim 24 further comprising the step of performing a surgical step on the stroma.

30. The method of claim 29 where the surgical step comprises reshaping the stroma.

31. The method of claim 30 further comprising the step of replacing an epithelial flap on the stroma.

32. The method of claim 31 further comprising the step of placing an ocular lens on the stroma.

33. The method of claim 32 further comprising the step of replacing an epithelial flap on the stroma.

34. The method of claim 25 where the step of applying a mechanical force comprises a step of forming an epithelial pocket or flap.

35. The method of claim 34 further comprising the step of placing an ocular lens on the stroma beneath the epithelium.

36. The structure formed by the method of claim 35.

37. A method for changing the vision of an eye having an anterior corneal surface and an epithelial tissue layer, the method comprising the step of:
placing a oscillating epithelial delaminator member of any of claims 1-12 beneath the epithelial tissue layer,
separating from the anterior corneal surface, a substantially continuous epithelial layer having a portion connected to the corneal surface, introducing an implant onto the corneal anterior surface, and
placing the attached epithelial tissue onto the implant.

38. The method of claim 37 where the step of introducing an implant onto the corneal anterior surface comprises introducing an ocular device comprising a synthetic polymer onto the uncut corneal anterior surface.

39. The method of claim 37 wherein the step of separating the substantially continuous epithelial layer produces an epithelial tissue layer containing substantially no corneal tissue.

40. The method of claim 39 wherein the step of separating produces an epithelial tissue flap containing substantially no corneal tissue.

41. The method of claim 39 wherein the step of separating produces an epithelial tissue pocket where the separated epithelial tissue contains substantially no corneal tissue.

42. The structure produced by the method of claim 37 comprising the implant in contact with the epithelial tissue and the corneal anterior surface.

43. The structure produced by the method of claim 38 comprising a synthetic polymer ocular device in contact with the epithelial tissue and the corneal anterior surface.

44. The structure produced by the method of claim 39 comprising the implant in contact with the epithelial tissue and the corneal anterior surface.
